(19) United States
(12) Patent Application Publication
(10) Pub. No.: US 2008/0317820 A1
(43) Pub. Date: Dec. 25, 2008

(54) Delivery System

(75) Inventors: Michael John Rathbone, Hamilton (NZ); Wade Jeffrey Mace, Hamilton (NZ); Keith James Ellis, Armidale (AU)

Correspondence Address: KREMBLAS, FOSTER, PHILLIPS & POLLICK, 7632 SLATE RIDGE BOULEVARD, REYNOLDSBURG, OH, 43068, US

(73) Assignee: INTERAG, Hamilton (NZ)

(21) Appl. No.: 12/091,805

(22) PCT Filed: Oct. 26, 2006
(86) PCT No.: PCT/NZ2006/000276

§ 371 Date: Aug. 26, 2008

(30) Foreign Application Priority Data
Oct. 28, 2005 (NZ) 543314

(51) Int. Cl. A61K 9/24

(52) U.S. Cl. 424/438

(57) ABSTRACT

A control release device for the delivery of active components, the device including; a rigid housing containing at least one discrete aperture therein, and a driving substance containing the active component(s) placed within the housing, characterized in that the driving substance swells in the presence of fluid, driving the substance and active components out of the housing through the apertures.

### TECHNICAL FIELD

This invention relates to a delivery system.

Specifically this invention relates to the delivery of active compounds to the rumen of animals.

### BACKGROUND ART

It is well understood, especially in the veterinary and animal treatment industries that it is often beneficial to have a long term continuous low dosage of active components being administered to an animal. This can provide significant advantages over the considerable up and down changes in concentration which are observed when discrete doses of active components are administered.

One reason to avoid high concentrations, or concentration changes are that some active compounds are toxic at high concentrations.

Alternatively, in some instances, high concentrations of active components may not be required to treat the condition. Instead, a continuous low dosage may be sufficient.

Controlled release devices are well known for animal treatment.

One very common form of a controlled release device is the bolus. A bolus is commonly in the form of an elongated cylinder designed to slowly dissolve in the rumen of the animal. Boluses are generally delivered into the rumen by use of a bolus applicator which delivers the bolus to the top of the animals esophagus, after which it is swallowed by the animal.

Boluses and other controlled release devices allow a discrete mechanism of delivery of a particular substance with a known release profile, wherein the amount of active agent which is delivered can be accurately known. This makes treatment and the analysis of the affects of treatment of the animal much more precise.

A bolus is usually comprised of a solid matrix coated in an impervious material having at least one opening through which the active material can be released. This prevents premature activation until the bolus is within the animals digestive tract, where it is desirable for the active component to be released.

Controlled release devices release their contents gradually over a period of time.

This mechanism saves considerable amount of labour and expense by allowing active ingredients to be delivered in one application, but to act over a period of time.

For example, most agricultural practices have large numbers of animals requiring treatment. Traditional delivery mechanisms such as drenches and injections require frequent applications as their effect may be short lived. It can be seen that frequent applications multiplied over a large number of animals results in a significant amount of labour, time and expense. Controlled release devices on the other hand require a single application per animal to last a significant period of time. The savings in labour and expense are therefore considerable.

The use of a substance or mechanism driving a controlled release devices via driving the active component(s) out of the device are known, however all current available devices make use of the following:
A device with separate compartments for the driving mechanism and the active components, and
Permeable walls adjacent to the compartment holding the driving substance (if this is driven by the fluid in a fluid containing environment (such as an animal's stomach) and expanding to drive the release of active components from the device. These devices therefore make use of the physical expansion of the driving substance to push the active components out of the device

The compartmentalisation of these devices means that these devices have more parts, resulting in higher manufacturing costs. The greater complexity also increases the chances of problems in controlling the rate of release accurately.

Examples of devices as described above include the following NZ patents;

New Zealand Patent No. 225058 describes a drug dispenser comprising a rigid housing and a fluid activated driving member. In this case the driving member is positioned within a separated portion of the housing, specifically the end of the housing opposite the opening through which the active components are to be released. The housing adjacent to the driving member is permeable to the fluid in the fluid containing environment, whereas the rest of the housing is not. When the fluid activated driving member is activated by the presence of fluid it pushes discrete drug units longitudinally along the housing and out of the outlet positioned at the end of the device.

New Zealand Patent No. 230872 describes a similar device wherein there is a housing which is separated into two portions one containing the beneficial agent(s) the other an osmagent or polymer which swells in the presence of fluid, causing a driving force to act upon a partition which pushes the beneficial agent out of the dispenser.

New Zealand Patent No. 237384 describes a similar mechanism however the driving substance may be in the form of an osmotic pump.

New Zealand Patent No. 232078 describes use of a dispensing device powered by a fluid activated driving member, being hydrogel, which is mixed with the active component(s).

In New Zealand Patent No. 232078 the hydrogel is coated with a coating comprising at least one water permeable polymer. In this case it is stated that it is not essential for the polymer to be semi permeable, examples given for the coating including cellulose acetate, silicon rubber, cellulose nitrate, polyvinyl alcohol, cellulose acetate butyrate, cellulose succinate, cellulose laurate, cellulose pellmate to name a few.

The active component(s) are released from the device through pores in the coating. The pores are preferably formed in the coating via porosigens in situ. However the pores may also be formed via other known methods such as mechanical or laser driven methods once the coating has been applied to the hydrogel.

The specification states that the hydrogel should be of a sufficient molecular weight that substantially no hydrogel is capable of leaving the device through the pores (page 8, lines 28 to 30).

New Zealand Patent No. 232078 also discloses that the device may instead of or as well as the pores contain one or more holes in the coating, or through the device, made by standing methods such as mechanical, sonic or laser drilling.

There are a number of disadvantages with the above disclosed device, including the following:

One major disadvantage is that the coating does not provide any structural rigidity to the device which is therefore susceptible to exterior physical forces. This lack of structural rigidity may lead to damage during transport or storage, or be a disadvantage if administered to an animal without further protection. Packaging and handling to prevent structural damage may increase the time and cost of packaging and transporting same.

Another significant disadvantage is that the coating requires a specialised coating process and associated machinery. This can significantly increase the cost and time required to manufacture the device.

If the pores are formed in situ it may also be hard to control the formation of same and the resulting release rate. Therefore another major disadvantage is that no control can be exerted over the exact number, size, size range or distribution of pores over the surface of the device.

Having a coating as described above also does not allow easy optimisation of the release rate other than altering the formulation of the tablet or mixture containing the beneficial components.

A further disadvantage is that the hydrogel is preferable retained within the coating, with the active components dissolving/moving out of the device through the pores into the environment it is positioned. A disadvantage of this is that the rate of release of the active components is limited by their diffusion rate through the expended hydrogel and out of the pores, leading to a slow release of same.

NZ 232078 also mentions that holes may be present in addition or in place of the pores, however these appear to be very general. NZ 232078 discloses holes which are made in the coating after the coating has been applied to the ‘tablet’ or mixture of beneficial agent and hydrogel. Thereby introducing additional steps into the manufacturing process and compounds or machinery to produce same, again this will increase the cost and time required for manufacture.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a control release device for the delivery of active components, the device including:
a housing containing at least one discrete aperture therein,
a driving substance containing at least one active component placed within the housing,
   characterised in that the driving substance swells in the presence of fluid, driving the substance and active components out of the housing through the aperture(s).

According to another aspect of the present invention there is provided a method for delivering active components via a control release device, the device including:
a housing containing at least one discrete aperture therein,
a driving substance containing at least one active component placed within the housing,
   the method characterised by the steps of
a) placing the driving substance containing the active component(s) into the housing,
b) administration of the control release device to an animal, or other environment for use,
c) the discrete apertures allowing the driving substance to come in contact with fluid in the environment causing the driving substance to swell,
d) the swollen driving substance exuding out of the discrete apertures, carrying the active component(s) with it,
e) the driving substance then being dissolved or eroded away releasing the active component(s) into the environment.

According to a further aspect of the present invention there is provided a housing, including:
at least one discrete aperture
   characterised in that the housing is configured to receive a driving substance containing at least one active component for delivery to an environment of use through the aperture(s).

The present invention may be used to provide at least one active component to any environment which contains a fluid capable of activating the driving substance, and to which an active component is wanted to be released into over time.

In a preferred embodiment the control release device may be one that can be used to deliver active components to the digestive system of an animal, such as the rumen.

Throughout this specification the term digestive system should be taken to refer to the gastrointestinal tract, including the stomach, the small intestines and the large intestines.

However, this should not be seen as limiting as it should be appreciated that it is possible to use the present invention to deliver active components to other positions of the animal, for example intravaginally.

Alternatively, the present invention may also be used to deliver active component(s) to systems/environments which contain a fluid to which an active component is wanted to be released into over time. One example of this may be use of the present invention to deliver components to the cistern of a toilet.

Throughout this specification the control release device will be referred to in respect to the delivery of at least one active component to an animal.

In a preferred embodiment the animal may be a ruminant, such as cattle or sheep, however this should not be seen as limiting as the delivery device may also be used for any other animal including humans.

In one particularly preferred embodiment the housing may be rigid, and shall be referred to as such herein.

Throughout this specification the term housing should be taken as meaning a rigid container into which the driving substance and active component(s) are placed.

Throughout this specification the term rigid in respect of the housing should be taken as meaning the housing is such that it will hold its own shape before it is filled with the driving substance and an active component(s).

Having a rigid housing provides physical protection to the driving substance and active components during storage and transport, preventing damage to same before administration. It also provides protection during administration.

In a preferred embodiment the housing is impermeable to fluid, except through the aperture(s). This means that the driving substance comes into contact with fluid which has entered the device through the aperture(s) and when it is activated may expand through same into the environment of use.

Throughout this specification the term impermeable shall be take as meaning not permitting the passage of fluid through a substance/material. However, depending on the material used to make the housing the passage of gas may be permitted. For example many plastics are permeable to gas.

In a preferred embodiment the rigid housing may be made of a material which is non-toxic, does not react with either the driving substance or the active component(s) and provides sufficient rigidity before administration to the animal.

The housing must possess sufficient strength to resist the physical stress incurred during administration and impelled upon the housing once in the environment of use, such as the rumen of an animal.

The housing may be designed to either break down internally or to be excreted by the animal. The mechanism of removing the housing may be controlled by the material used to make same. Internal break down of the housing can be achieved by using a biodegradable material in the manufacture of same, whereas to be excreted non-biodegradable material would be used.

If internal break down were to be utilized, then it would be preferable if the break down of the housing took significantly longer than the release period of the active components. For example, if the release period was three months, a break down rate of the housing may be twelve months. However this should not be seen as limiting as in some cases it may be desirable to have the break down period similar to that of the release rate.

In a preferred embodiment the rigid housing may be made out of plastic, and shall be referred to as such herein. However this should not be seen as limiting as any material which has the desired properties may be utilized in the present invention.

Some examples of plastics which may be used are: nylon, polyethylene and propropylene. However this should not be seen as limiting as other plastics (biodegradable or non-biodegradable) may be utilised.

In a preferred embodiment the plastic will have a thickness which provides sufficient strength to the housing to resist the physical stress incurred during administration and impelled upon the housing once in the environment, such as the rumen of an animal.

The thickness of the plastic will depend upon the rigidity and inherent strength of the plastic that is used to manufacture the housing.

In a preferred embodiment the housing may be substantially cylindrical in shape, and may have a substantially circular or oval longitudinal cross sectional shape.

This shape removes the presence of sharp corners which may snag on or damage the inside of the intestinal tract, and allows for easy fitting of the driving substance/active component when in the preferred form of tablets (as discussed below). However this should not be seen as limiting as variations on this shape may be utilised with the present invention.

In a preferred embodiment the housing may be of dimensions suitable to hold sufficient active components and driving substance to deliver same for the treatment period, and to retain the device in the digestive system. If the device is to be maintained in the digestive system due to its geometry then it must be of a sufficient length to ensure same. If the device is to be maintained in the digestive system due to density, then it must have a sufficient density to ensure same. The length or density required to maintain the device is dependant on the type of animal it is to be administered to, and would be able to be easily calculated by one skilled in the art.

In a preferred embodiment the rigid housing may also include a wing, or pair of wings, which help to maintain same within the digestive system of an animal. The use of wings, and variations in same to maintain a control release device in the correct position within the intestinal tract would be well known to one skilled in the art. One skilled in the art would be easily able to adapt known wings for use with the present invention.

The term a wing should be taken as including one or more protrusions extending from the housing, or end of same, designed to help maintain the housing within the digestive tract.

In preferred embodiments the wing(s) may be held alongside the housing during administration. This may be by a dissolvable or paper means, or any other means know to one skilled in the art.

In a preferred embodiment the housing contains at least one discrete aperture therein.

Throughout this specification the term aperture should be taken as meaning an opening or gap through which the driving substance and active component(s) can pass.

In a preferred embodiment the aperture(s) may be located along the longitudinal sides of the housing.

In some embodiments there may also be apertures on at least one end of the housing. This increases the efficiency of delivery of the active component to the environment of use.

However, this should not be seen as limiting as there may be variations in the number and arrangement of aperture(s) in respect to one another and the housing.

The size and number of apertures can be designed to provide the desired release rate. The larger the aperture(s) and/or the greater the number of same, the greater the surface area of the driving substance/active component(s) in contact with the environment, and thus the quicker the release rate of the active component(s). This is due to the increased driving force and the extrusion/dissolution of the driving substance/active components, thereby increasing the release rate of the active component(s) into the environment of use.

Therefore to increase the target release rate the housing may have either, or both: an increased number of apertures, or apertures of an increased size.

It should be recognised that having a higher number of smaller apertures will retain maximum structural rigidity of the housing, therefore less material, or material of a weaker nature may be utilized in the manufacture of same. However, it should be noted that apertures must be of a sufficient size to ensure the rumen fluid will come into contact with the driving substance, leading to the swelling of same.

In one preferred embodiment the housing is configured with at least one row of apertures down the side of same, this provides rapid delivery of the active component to the environment of use.

In a particularly preferred embodiment the housing may have two rows of apertures down opposing sides of the housing. However this should not be seen as limiting as other configurations may also be envisaged. For example, for a slower release rate only one or two apertures in total may be utilised.

In a preferred embodiment the active component may be any active component which has a beneficial action in the environment of use and can be formulated into a controlled release dosage for use in the present invention and administrated via same.

Examples of active component(s) include, but are not limited to minerals, vitamins, trace elements and other beneficial or treatment substances to be administered to an animal.

In a preferred embodiment the driving substance may be a swellable material which swells on contact with a fluid. In the case of the present invention being used in the digestive system of an animal it is important that the driving substance (and/or active components) is not damaged by the environment of same, such as the low pH.

In a preferred embodiment the driving substance may be a hydrogel, and shall be referred to as such herein.

Hydrogels are polymers which are capable of swelling in the presence of a fluid due to absorption of fluid into the hydrogel matrix when the delivery device is used for administration to an animal's digestive system, the fluid will be digestive fluid(s).

The present invention may make use of one, or a combination of two or more hydrogels as the driving substance.

Hydrogels which could be used with the present invention include any known, or yet to be developed hydrogels and would be known to those skilled in the art.

In a preferred embodiment the hydrogel or combination of hydrogels used will allow high loading of the active component per volume of hydrogel. However this should not be seen as limiting as in some instances high loading may not be desired, for example when very low delivery rates over a long period of time are desired.

In one preferred embodiment the hydrogel may be polyethylene oxide (PEO). A PEO matrix is very porous and therefore allows high loading of the active component.

In an alternative embodiment the present invention may include a mixture of two or more hydrogels, for example PEO plus tragacanth gum, HPMC or xanthan gum.

As well as swelling/expanding hydrogels also undergo dissolution in the presence of fluid, thereby breaking down into constituent parts. As the hydrogel and active component mixture extrudes from the housing it undergoes dissolution in the digestive fluid, leading to the release of the active component.

Throughout this specification the term dissolution shall be taken to mean the disintegration of the hydrogel/active component mixture and dissipation of same.

Alternatively the active component may diffuse out of the hydrogel as the hydrogel forms a porous matrix. The active might diffuse through the pores out of the hydrogel.

In preferred embodiments the active component may be retained within the hydrogel matrix, this allows easy release of the active component when the hydrogel erodes, or when dissolution occurs. This is dependent upon the binding affinity of the active to the matrix polymer(s) and the solubility of the active. If the active binds to the matrix polymers via reversible chemical bonds (hydrogen bonds, ionic bonds, dipole-dipole bonds or Van-der-Waal bonds), the active will remain mostly in the matrix until it undergoes dissolution, meaning a low contribution of diffusion to the drug release. If the active however has no or low binding affinity to the polymer and is soluble in the environmental fluid, diffusion might contribute in a higher extent to the release of the active. Diffusion cannot occur to insoluble compounds. The contribution of erosion/dissolution and diffusion to the drug release is therefore dependent upon the chemical properties of the active and the polymer(s).

Whether the active component is released from the hydrogel via erosion, dissolution or diffusion depends on the release rate and the position of the active component in question. It is likely that in many cases the release mechanism may be a combination of erosion/dissolution and diffusion. Once administered the hydrogel will come into contact with fluid and swell, leading to extrusion out through the apertures, dissolution will then occur, however later in the release profile the matrix inside the housing will be more and more diluted, so that the fluid entering through the apertures will then lead to dissolution or dissolving within the housing.

In a preferred embodiment the rigid housing is made separately to the active component(s) and hydrogel mixture to be contained within same.

These has the significant advantage that the tablets (see below) of hydrogel and active components can be manufactured and undergo quality control separately, and prior to being assembled/placed into the housing.

In a preferred embodiment the hydrogel and active components may be made into a tablet configured to fit inside the housing and shall be referred to as such herein. However this should not be seen as limiting as other forms may also be utilized, such as a gel, paste or extrudate

Having the hydrogel/active component in a tablet form makes it easy to handle and to fill the housing. Having the hydrogel and active component(s) in a tablet matrix also ensures that the formulation is physically and chemically stable. Tablets also allow differing dosages to be provided within the housing by varying the number of tablets inserted into same or by varying the percentage composition of the active component(s) within the tablet matrix. Tablets allow well established and reproducible manufacturing processes to be used. These processes also allow variations in tablet sizes such as diameter and thickness to be easily accommodated.

In a preferred embodiment the hydrogel and active components are mixed into a uniform powder before being formed into tablets.

In some embodiments in addition to the driving substance and active component(s) the tablets may also include additional excipients.

Throughout this specification the term “excipient” shall be taken to mean an inactive or inert substance, which is not a medicinally active constituent.

The excipient is combined with an active component in order to produce a deliverable substance. The excipient may give the mixture of an active component and hydrogel increased consistency or form or provide additional stability or bulk. The excipient may help to manufacture the tablets.

In a preferred embodiment a number of tablets are “stacked” inside the housing one beside another. However, this should not be seen as limiting, for example one large tablet which fills the housing may be utilised.

Throughout this specification the term “stack” should be taken as meaning an ordered row of tablets which can then sit one beside the other inside the housing

The benefit of stacking a number of tablets inside the housing is the versatility. For example it is possible to have a constant concentration of the active component throughout the tablet stack. Alternatively it is easy to vary the concentration of active component within the tablet stack, for example increasing the concentration to accommodate an increase in animal weight due to growth. A further alternative is to incorporate multiple tablets, containing different active components in a housing.

A number of narrow tablets placed one beside the other are preferred over one long cylindrical tablet. This is due to the fact that cylindrical tablets are usually formed by extrusion rather than compression; this requires a much higher temperature and provides a more aggressive and damaging environment to the active ingredient. This may be detrimental to the active ingredient and lead to degradation or loss of bioactivity of same. This may therefore limit the type of active ingredient which can be used. However for some active components this method may be suitable.

In a preferred embodiment known techniques to produce tablets may be utilized with the preset invention, these would be well known to one skilled in the art.

The tablets may be of a variety of forms.

In one embodiment the tablet may be a solid tablet.

Alternatively hollow cored tablets of a “lifesaver” shape may be utilised to minimise the distance the active component travels but maximising the volume and area and therefore the active component delivered and rate of delivery. This increases the surface area of the tablet exposed to the environment without increasing the volume, thereby increasing the rate at which the active component is released. This results in maximum active component utilization with the minimum volume of excipient.

Alternatively tablets with an extruded core of PEO (or alternative swelling polymer) containing no active component or tablets with a second tablet core of PEO (or alternative swelling polymer) containing no active component may be utilised. Either of these cores could potentially incorporate a second active component.

A further alternative is the use of “fizzy” tablets incorporating compounds which generate a gas such as CO₂ to help deliver the active component to the environment.

In a preferred embodiment bicarbonate and citric acid may be used co-excipients to generate CO₂. The CO₂ generation can act as a driving substance in addition to, or in place of the hydrogel. The CO₂ generation helps to expel the active component from the delivery device.

Erosion or dissolution of the driving substance (hydrogel) releases the physically entrapped active components into the rumen or intestinal tract or other environment in which the control release device is present, which is therefore available for absorption either into the animal or to carry out the required reactions in same.

Progressive presence of fluid leads to the continual swelling of the driving substance through the discrete apertures of the rigid housing and into the environment, resulting in the subsequent release of active components over a time period.

When fluid comes into contact with the solid tablet, the hydrogel forms a gel, which swells out through the apertures in the housing releasing the active component(s) into the rumen environment.

The formulation may also be designed to give the desired release rate. This may be achieved by altering either the molecular weight of the hydrogel, or the percentage of same in the formulation.

Changing the molecular weight of the hydrogel will affect the swelling rate, swelling degree, viscosity, the amount of water in the swollen matrix, the porosity of the matrix and, the matrix structure. All these factors will affect the release rate. Lower molecular weight hydrogels have a faster release rate, whereas those with higher molecular weights have a slower release rate.

Increasing the percentage of hydrogel in the formulation will also decrease the release rate. This is due to the matrix being more viscous, therefore slowing down the erosion and dissolution of the matrix and the active component(s) will diffuse through same slower, resulting in a slower release rate. Experimentation has shown that increasing the percentage of hydrogel within the tablet formulation decreases the release rate, leading to an extended release profile.

In practice large changes in the release rate would be achieved via alteration of the housing design regarding the number and size of apertures, and fine tuning of the release rate would be achieved via alteration of the hydrogel/active component formulation.

There are therefore a number of factors which influence the rate of release of the active component(s) from the driving substance and rigid housing, these include the following:
aperture size, the aperture provides a constant surface for erosion to occur,
number of apertures,
the concentration of hydrogel (or driving substance),
the type of hydrogel (or driving substance) for example molecular weight of HPMC or PEO,
the type of active component(s),
whether the active component(s) interacts with the driving substance,

Secondary factors affecting the release rate of the active might be:
shape of apertures
thickness of the housing device
tablet properties (such as hardness) changes in the physiological environment (pH, temperature, . . . )

Advantages of the present invention over previous control release devices, include:
easy changes to the configuration of the housing and apertures to significantly change the release rate,
changing the formulation (for example by using a hydrogel with a different molecular weight) to fine tune the release rate,
having a very controlled and predictable release rate, which is linear over a long period of release,
having a separate housing allows optimisation of the release rate to be independent of the formulation of the tablet,
the rigidity of the housing provides increased physical protection to the tablets during storage and transport of the device,
it allows easy manufacture of the components separately,
having the tablets manufactured separately to the housing allows the tablets to undergo quality control separately and prior to assembly/placing into the housing,
the apertures are made in the rigid housing at the time of manufacture, not once the housing has been formed, this decreases the manufacturing costs and the labour or machinery required for same,
the tablets are introduced into the housing, not the housing formed around the tablets, this increases the flexibility of the delivery device, as a number of tablets of differing concentrations or containing differing active components may be introduced easily, without having to change any of the manufacturing requirements.

### BRIEF DESCRIPTION OF DRAWINGS

Further aspects of the present invention will become apparent from the following description which is given by way of example only and with reference to the accompanying drawings in which:

FIG. 1 a and b show schematics of the rigid housing and tablet for same according to a preferred aspect of the present invention;

FIG. 2 a and b show schematics of a two hole configuration of the control release device according to one aspect of the present invention;

FIG. 3 a and b show schematics of the rigid housing, including three rows of apertures, according to another aspect of the present invention;

FIG. 4 a and b show schematics of the rigid housing, including six rows of apertures, according to another aspect of the present invention;

FIG. 5 a and b show schematics of the rigid housing, including nine rows of apertures, according to another aspect of the present invention;

FIG. 6 a to f show schematics of how the hydrogel/active component extends out of the housing when the hydrogel is activated by the presence of fluid; and

FIG. 7 shows the housing according to one aspect of the present invention adapted for use in pigs.

### BEST MODES FOR CARRYING OUT THE INVENTION

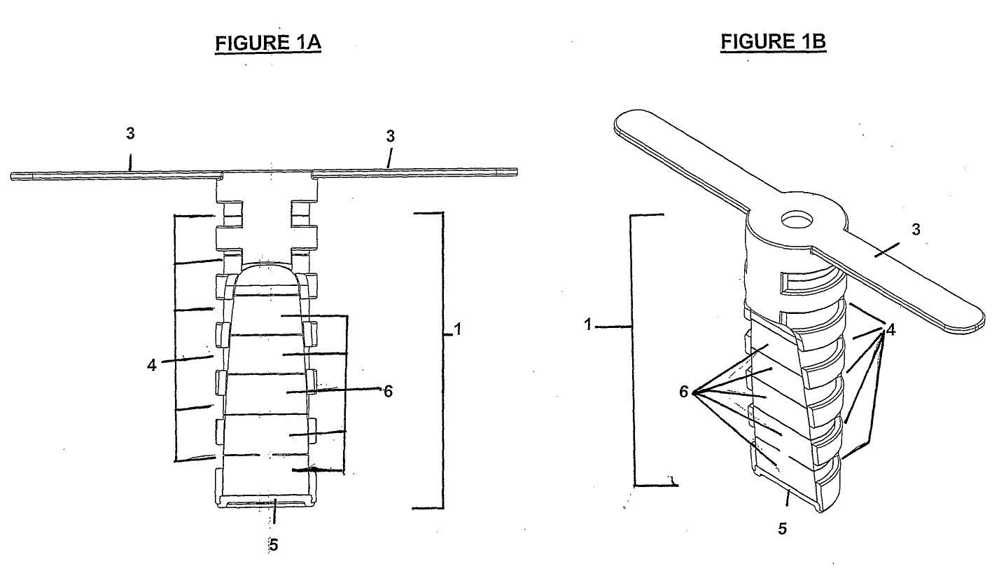

FIG. 1 shows a schematic of one variation of the delivery device configured to be maintained in the rumen of an animal according to the present invention. FIGS. 1a and b show a delivery device which includes wings to maintain same in the rumen. In some alternative embodiments the device may be maintained in the rumen by a weighted core (in this instance, the device would not include wings).

FIGS. 1a and b shows a rigid housing, generally shown by (1). The rigid housing has a number of apertures, looking like slots (4), and a pair of wings (3) attached to one end of the housing to help maintain the housing/control release device in the intestinal tract of the animal.

The discrete apertures (4) allow the tablet(s) of hydrogel and active components to extend through same as the hydrogel swells in the presence of fluid. The swollen hydrogel and active components are pushed through the apertures and is then acted upon by intestinal fluids and erodes/undergoes dissolution/dissolving to release the active component(s) into the intestinal tract for absorption.

The only openings in the housing (1) (once ready for administration) are the apertures (4).

The housing also includes an end cap (5) which is applied once the tablet(s) of the hydrogel/active components have been introduced to the housing.

The housing (1) is shown in a cut away view showing tablets (6) placed along the longitudinal length of the housing.

In preferred embodiments the housing may also include a piece of compressible material at one end of the rigid housing, for example, a piece of sponge. Preferably this is positioned at the opposing end of the housing from the end to which the tablet(s) are introduced. The compressible material ensures that the tablets fit snugly into the housing, by pushing the tablets together and substantially preventing any gaps between adjacent tablets. Gaps between adjacent tablets may lead to an undesired non-linear release rate of the active ingredient(s).

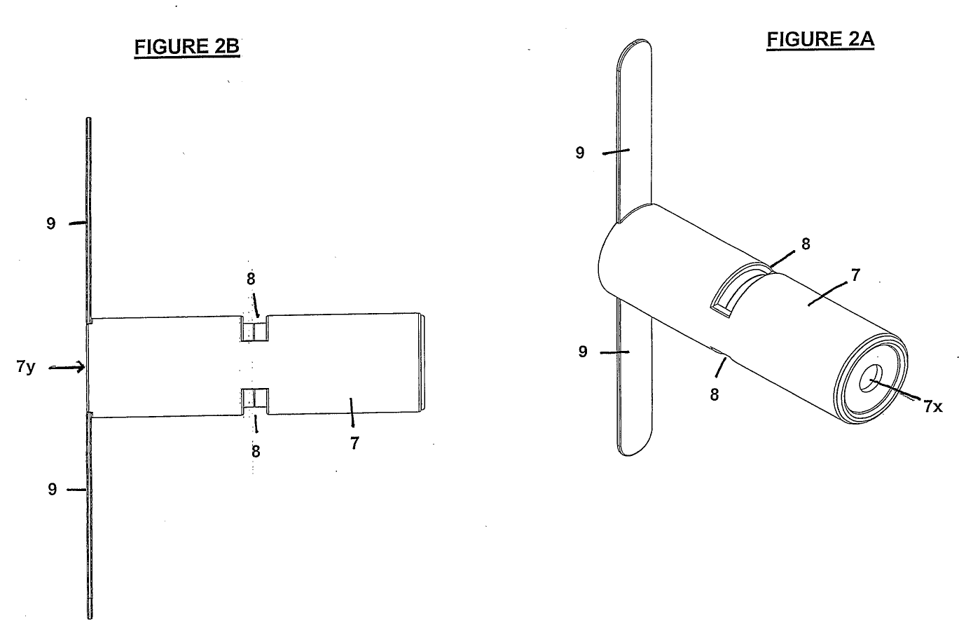

FIG. 2 shows a schematic of a housing containing an aperture at either end and two apertures around the centre circumference. In both the housing is shown by (7), the aperture(s) by (8) and the pair of wings by (9). This configuration, with two apertures on opposite sides of the housing is preferred. This is irrespective of the number of rows of apertures. Having opposing apertures ensure consistent release of the hydrogel/active component(s) when the device is in the digestive tract of the animal.

FIG. 2b also shows an aperture in the end of the housing (7x). An aperture may also (or instead) be present in the other end of the housing (not shown in this Figure, but indicated by 7y).

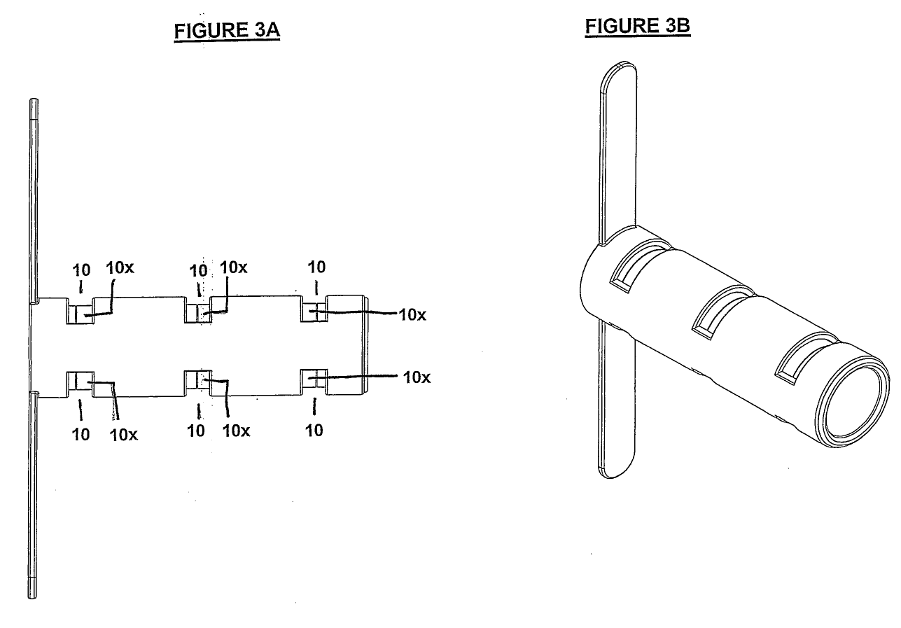

FIG. 3 shows a schematic of housing with three rows of apertures (10).

This is in the preferred configuration of a row of apertures down either side of the housing. The tablets within the housing are also shown (10x)

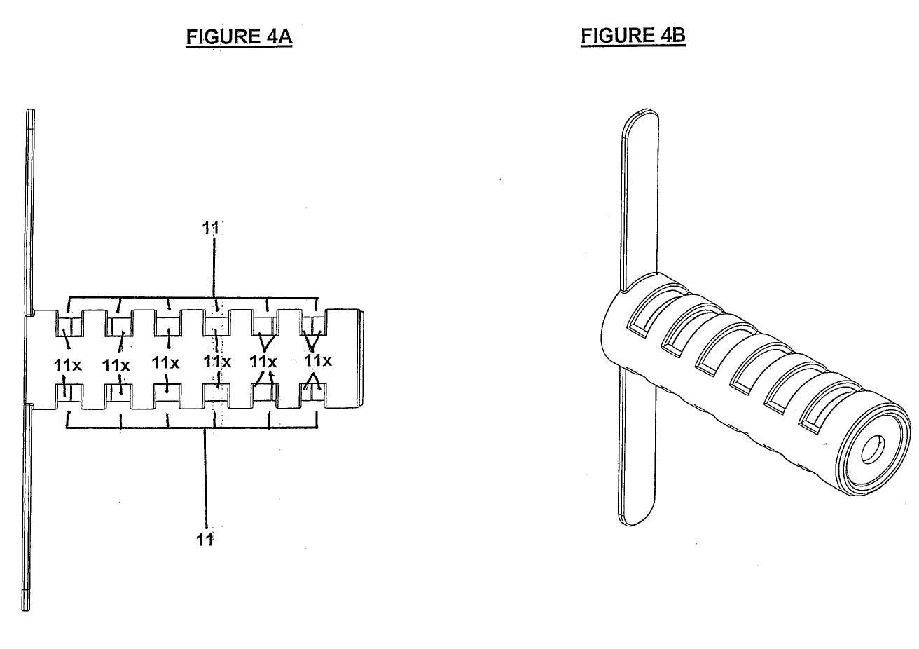

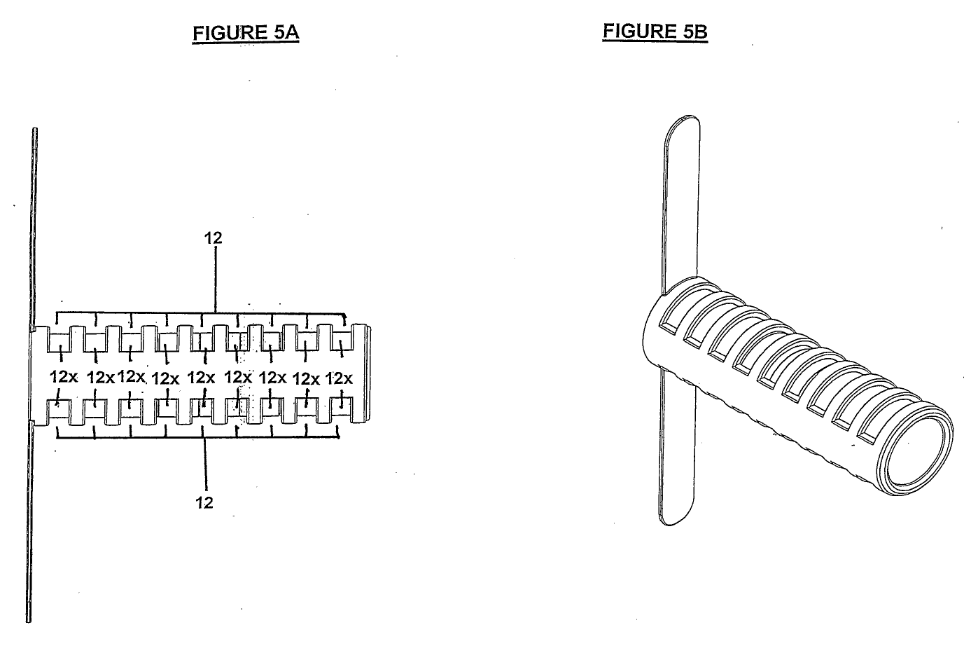

Similarly FIGS. 4a and b show a similar schematic of a housing with six apertures (11) on either side of the housing, and tablets (11x). FIGS. 5a and b shows nine apertures (12) on either side of the housing, and tablets (12x).

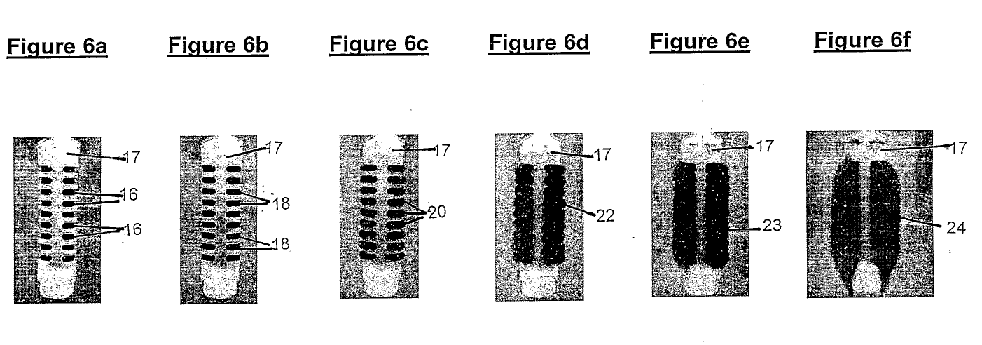

FIG. 6a to 6f shows a sequence of the activation of the hydrogel and the extrusion of same (along with the active components) through apertures in the housing (17) and into the destination environment. FIG. 6c to 6f show the hydrogel extending out of the apertures whereon it can be acted upon by the fluid in the intestinal tract, this erodes and dissolves the hydrogel, releasing the active components into the intestinal tract.

FIG. 6a shows the tablet (16) within the housing (17).

FIG. 6b shows the hydrogel of the tablets beginning to expand. At this stage the hydrogel (18) has expanded to the edge of the housing (17).

In FIG. 6c the hydrogel (20) has expanded out of the housing (17).

FIG. 6d to 6f show the hydrogel (22), (23) and (24) expanding further out of the housing (17).

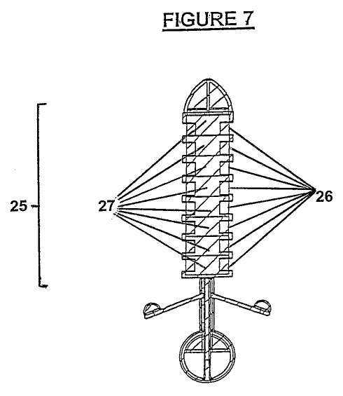

FIG. 7 shows a schematic of the housing, generally shown by (25) with two rows of eight apertures (26) down opposing sides of the housing (25), adapted for use with pigs, the view shown is a cut away view showing the interior of the housing and tablets (27).

### Experimental Results

### 1. Sodium Salicylate Release Experiments—In Vitro

### 1.1 Methodology

Sodium salicylate was used to study the release rate from devices according to the present invention.

For sodium salicylate release studies, deionised water was used as the release media. The devices were immersed in sufficient release media to ensure continued wetting of the tablets through the aperture(s) in the devices, and to ensure that at all times the devices were in conditions under which release could occur (with respect to the active, sodium salicylate).

At each sampling, a sample of the release media was removed for analysis, and the devices placed into fresh release media. During the release test, the devices were gently agitated using an orbital shaker, so as to ensure even release from the devices, and to homogenise the release media (thereby ensuring release conditions are maintained). This also simulated movement of the device which may be expected in environments of use, such as in the digestive system of an animal.

The samples were run in triplicate. The exception being the determination of the release from the single orifice devices, where five samples were run for the first 14 days, four samples for the following 14 days, and three samples for the remainder.

The quantity of sodium salicylate released was determined by UV-Vis spectroscopy at 295 nm.

### 1.2 Methodology for Vertical Swelling Experiment

A tablet of the required composition was placed in the bottom of a tube the same diameter as the tablet. 5 mL of deionised water was placed on top of the tablet, and the swelling of the tablet monitored with reference to a scale on the side of the tube. Each composition was repeated in triplicate.

1.3 Methodology for Extrusion of Rod and Tablet from Single Orifice Device (6 mm)

The device was suspended in an release media (deionised water) and allowed to swell. At each sampling any PEO that had swelled out of the orifice was scarped off into a separate vessel and dried. The dry weight was then recorded.

### 1.4 Results

Graph 1 shows the difference that the number of apertures (slots) can have on the release rate for the device. Three, six and nine rows of apertures in the housing are compared, for each the apertures were the same size.

Graph 1 shows that complete release from the 3, 6 and 9 slot devices is attained at 5, 7 and 14 days respectively. The initial release rate from the 3, 6 and 9 slot devices is 24%, 19% and 10% of total active per day, respectively. (The active in question was sodium salicylate, the tablet matrix contained 5% active by weight, with sucrose as an excipient and magnesium stearate as a lubricant for the tablet making process).

The initial period used to calculate the release rate was up to (and including) the 3^{rd} day for the 3 slot device, the 5^{th} day for the 6 slot device, and the 8^{th} day for the 9 slot device (the initial linear portions of the curves).

Near linear release is achieved for ≧80% release of the total active. The lines on Graph 1 represent an average of the data points shown on the graph.

Graph 2 shows the difference that the concentration of PEO within the tablets can have on the release from the device.

Graph 2 shows the release of the active from tablets containing either 7.5% or 25% PEO, held within a six slot device. (The active in question was sodium salicylate, the tablet matrix contained 5% active by weight, with sucrose as the excipient and magnesium stearate as a lubricant for the tablet making process).

The initial release rate from the 7.5% PEO tablets was 33% per day, compared to 19% per day for the 25% PEO tablets. The initial period used to calculate the release rate was up to the 3^{rd} day for the 7.5% PEO tablets and the 5^{th} day for the 25% PEO tablets.

Near linear release is achieved for ≧80% release of the total active. The lines on Graph 2 represent an average of the data points shown on the graph.

Graph 3 shows the difference that the concentration of PEO within the tablets can have on the release from a single aperture device.

Graph 3 shows the release of sodium salicylate (the active) from tablets containing varying quantities of PEO (5%, 7.5%, 10%, 15%, 20% and 25% composition by weight). The tablet matrix contained 5% active by weight, with sucrose as the excipient and magnesium stearate as a lubricant for the tablet making process.

The tablets were contained within a device comprising one aperture at the end (similar to that shown in FIG. 1B).

Each line (point) on the graph represents the average of three (or more) experimental data points.

Complete release from the 5% PEO tablet is achieved after 40 days.

The initial release rate from the 5%, 7.5%, 10%, 15%, 20% and 25% tablets is 2.9%, 2.1%, 1.5%, 1.2%, 1.1% and 0.95% of total active per day, respectively. The initial period used to calculate the release rate was up to (and including) the 28^{th} day.

Graph 4 shows a range of possible release profiles that can be achieved through varying the composition of the tablet or the number of apertures or orifices (located in the ends of the device) in the device.

Graph 5 shows the difference that the composition of PEO in the tablet can have on the rate of swelling of the tablet.

In Graph 5, the tablets consisted of 10%, 20%, 40%, 80% or 99% PEO, with the remaining matrix consisting of lactose as the excipient, and 1% magnesium stearate as a lubricant for the tablet making process.

The tablets swelled at 1.05%, 0.86%, 1.1%, 1.25% and 1.33% per hour respectively, after an initial period of rapid swelling. Data from 72 hours onwards was used to calculate the rate of swelling, as after this time the rate of swelling was linear.

Each graph point is the average of three experimental data points.

Graph 6 shows the rate at which PEO swells (is exuded) from the orifice of a single orifice device, comparing the amount of a PEO exuded form a tablet to the amount exuded from a solid rod (formed by swelling of the PEO).

Graph 6 shows the rate at which PEO swells (is exuded) from the orifice of a single orifice device, comparing the amount of a PEO exuded form a tablet to the amount exuded from a solid rod (formed by swelling of the PEO). The rod swells (is exuded) at a rate of 14 mg/day compared to 28 mg/day for the tablet.

### 2. Animal Studies—In Vivo

Animal trials are currently on-going. They started on 14 Jun. 2006, and are expected to be completed by January 2007.

An accompanying in vitro drug release study is also currently on-going. This started on 28 Jun. 2006, and is expected to be completed by February 2007.

The below details of these studies is therefore limited to preliminary data from 2, 4 and 8 weeks of the in vitro and in vivo studies.

### 2.1 Aim

The purpose of the animal trials is to determine and define drug release performance of the rumen delivery system in the rumen environment.

The purpose of the drug release study is to demonstrate for three different drugs with varying physiochemical properties, linear drug release over 16 or 32 weeks, and any factors affecting same.

The drug release study also demonstrates the impact of key parameters on dry release, including aperture size, PEO concentration and HPMC concentration.

### 2.2 Methodology: Animal Trial (In Vivo)

The trials were performed in fistulated cattle.

The following compounds were used as model drugs:
MgSO4: a mineral, water-soluble drug/compound,
Kaolin insoluble, and
Sodium Salicylate, an organic compound which is water-soluble.
The devices containing the above drugs were inserted into the fistulated rumen at t=0 and withdrawn after 2, 4, 8, 12 and 16 weeks.
The residual drug content was analyzed and the % drug release was calculated using the following equation:

% drug release=(initial drug content−residual drug content)/initial drug content*100%
Each variant was determined in duplicate or quadruplicate at each time point to determine the robustness of the data.

Validated analytical methods were used to determine the residual drug content, as follows:
MgSO4: UV-spectrophotometric method (with dihydroxyazobenzene)
Kaolin: gravimetric method
Sodium salicylate: UV-spectrophotometric method

The drug release performances of the variants shown in table 1 were investigated in the animal trial:

TABLE 1
Variants used in animal trials (hydrogel/active component)

| | | | Target |
|---|---|---|---|
| Variant | Aperture | | release |
| ID | size | Nominal composition | (days) |
| #1 | 2 × 1 mm | MgSO4 50%; PEO 20%; Lactose 30% | 100 d |
| #2 | 2 × 3 mm | MgSO4 50%; PEO 20%; HPMC 30% | 100 d |
| #3 | 2 × 5 mm | Kaolin 50%; PEO 20%; Lactose 30% | 100 d |
| #4 | 2 × 3 mm | NaS 50%; PEO 20%; Lactose 30% | 100 d |
| #5 | 2 × 4 mm | Kaolin 50%; PEO 20%; Lactose 30% | 200 d |
| #6 | 2 × 2 mm | NaS 50%; PEO 20%; Lactose 30% | 200 d |

TABLE 2
Detailed composition of the tablet formulation for variant #1:

| | Actual | |
|---|---|---|
| | composition | |
| Substance | (% w/w) | Function |
| MgSO4 (dried) | 49.5% | Water-soluble mineral model |
| | | drug |
| PEO WSR 303 | 19.8% | Swelling excipient |
| Lactose monohydrate | 29.7% | Binder |
| Magnesium stearate | 0.99% | Lubricant (for tabletting) |

TABLE 3
Detailed composition of the tablet formulation for variant #2

| | Actual | |
|---|---|---|
| | composition | |
| Substance | (% w/w) | Function |
| MgSO4 (dried) | 49.14% | Watersoluble mineral model |
| | | drug |
| PEO WSR 303 | 19.66% | Swelling excipient |
| HPMC K100M | 29.48% | Swelling/retarding polymer |
| Magnesium stearate | 1.23% | Lubricant (for tabletting) |
| Aerosil | 0.49% | Glidant agent (for tabletting) |

TABLE 4
Detailed composition of the tablet formulation
for variant #3 and #5:

| | Actual | |
|---|---|---|
| | composition | |
| Substance | (% w/w) | Function |
| Kaolin | 48.44% | Insoluble model drug |
| PEO | 19.37% | Swelling excipient |
| Lactose | 29.06% | Binder |
| PVP | 1.19% | Granulation agent |
| Magnesium stearate | 1.94% | Lubricant (for tabletting) |

TABLE 5
Detailed composition of the tablet formulation
for variant #4 and #6:

| | Actual | |
|---|---|---|
| | composition | |
| Substance | (% w/w) | Function |
| NaS | 47.85% | Watersoluble organic model |
| | | drug |
| PEO | 19.14% | Swelling excipient |
| Lactose | 28.71% | Binder |
| Magnesium stearate | 4.3% | Lubricant (for tabletting) |

### 2.3 Methodology In Vitro Drug Release

The drug release was tested in 200 ml water as release media at 39° C.±1° C. on a bottle roller apparatus (bottles rotate with 50±2 rpm)

At each sampling (weekly or every second week), the drug content was determined in the release media and the devices were placed into fresh release media.

### 2.4 Results

### 2.5 Na Salicylate Formulations—100 and 200 Day (Variant #4 and 6)

### 2.5.1 In Vivo Drug Release Na Salicylate Formulation (100 Days)

Drug release of the individual 4 replicates, mean drug release and standard deviation is shown in Table 6.

TABLE 6
In vivo release of Na salicylate (100 days)

| Time | Replicate 1 (% | Replicate 2 (% | Replicate 3 (% | Replicate 4 (% | Mean (% | |
|---|---|---|---|---|---|---|
| (weeks) | drug release) | drug release) | drug release) | drug release) | drug release) | SD |
| 0 | 0.0 | 0.0 | 0.0 | 0.0 | 0.0 | 0.0 |
| 2 | 10.9 | 10.1 | 10.2 | 11.2 | 10.6 | 0.5 |
| 4 | 22.1 | 23.1 | 22.2 | 22.2 | 22.4 | 0.5 |
| 8 | 40.4 | 42.5 | 41.3 | 41.4 | 41.4 | 0.9 |

Graphs 7 and 8 show diagrammatically the in vitro release of Na salicylate.

Graph 7a shows that linear drug release was observed for the first eight weeks of the trial, as expected.

Graph 7b, showing the individual drug release for the 4 replicas shows that there was very low variability between replications. This indicates the robustness of the drug release.

From the extrapolation of data it appears to be likely that the goal will be achieved, being zero-order drug release within 16 weeks, resulting in a constant active component concentration in the environment of release throughout the delivery period.

This graph shows perfect correlation between the in vitro and the in vivo drug release. The in vitro method seems to be indicative for the drug release of the sodium salicylate device in the cattle rumen.

### 2.5.2 In Vivo Drug Release Na Salicylate Formulation (200 Days)

Drug release of the individual 2 or 4 replicates, mean drug release and standard deviation is shown in Table 7.

TABLE 7
In vivo release of Na salicylate (200 day)

| Time | Replicate 1 (% | Replicate 2 (% | Replicate 3 (% | Replicate 4 (% | Mean (% | |
|---|---|---|---|---|---|---|
| (weeks) | drug release) | drug release) | drug release) | drug release) | drug release) | SD |
| 0 | 0.0 | 0.0 | 0.0 | 0.0 | 0.0 | 0.0 |
| 4 | 9.5 | 9.2 | — | — | 9.3 | 0.2 |
| 8 | 20.5 | 20.3 | 21.0 | 20.1 | 20.5 | 0.4 |

Graphs 10 and 11 show diagrammatically the in vitro release of Na salicylate.

Graph 10a shows that linear drug release was observed for the first eight weeks of the trial, as expected.

Graph 10b, showing the individual drug release for the 2 or 4 replicas shows that there was very low variability between replications. This indicates the robustness of the drug release.

From the extrapolation of data it appears to be likely that the goal will be achieved—being zero-order drug release over 32 weeks.

Graph 12 shows perfect in vivo/in vitro correlation.

### 2.5.3 Na Salicylate Formulation (100 Day)—Observations:

The pictures in Graph 13 show the dried and opened drug delivery devices. It was observed that the rate of swelling was greater than the rate of erosion, as no erosion took place inside the housing. A, B and C of Graph 13 show the observations for 2, 4 and 8 weeks respectively.

Erosion takes place outside of the apertures in the rigid housing, therefore, the drug release rate was controlled by the constant surface of the apertures.

### 2.5.4 Na Salicylate Formulation (200 Day)—Observations:

It was observed that the rate of swelling was greater than the rate of erosion, as shown in Graph 14 (pictures of dried and opened drug delivery devices). A and B of Graph 14 show the observations for 4 and 8 weeks respectively.

Erosion takes place outside of the apertures in the rigid housing; therefore, the drug release rate was controlled by the constant surface of the apertures.

### 2.5.6 Conclusions: Na Salicylate Formulation—100 and 200 Days

Drug release was linear so far, and shows very low variability. This was the expected behavior.
Promising linear release of Na salicylate for the entire 100 or 200 days of animal trial.

### 2.6 Kaolin Formulations—100 and 200 Day (Variant #'s 3 and 5)

### 2.6.1 In Vivo Drug Release Kaolin Formulation (100 Days)

Drug release of the individual 4 replicates, mean drug release and standard deviation is shown in Table 8.

TABLE 8
In vivo release of Kaolin (100 days)

| Time | Replicate 1 (% | Replicate 2 (% | Replicate 3 (% | Replicate 4 (% | Mean (% | |
|---|---|---|---|---|---|---|
| (weeks) | drug release) | drug release) | drug release) | drug release) | drug release) | SD |
| 0 | 0.0 | 0.0 | 0.0 | 0.0 | 0.0 | 0.0 |
| 2 | 8.7 | 11.4 | 9.8 | 10.3 | 10.1 | 1.1 |
| 4 | 18.8 | 21.7 | 20.0 | 20.6 | 20.3 | 1.2 |
| 8 | 66.9 | 71.3 | 75.5 | 73.6 | 71.8 | 3.7 |

Graphs 15 shows diagrammatically the in vivo release of Kaolin.

Graph 15a shows that there was an increase in release rate after 4 weeks, therefore linear drug release was not observed over the first eight weeks of the trial.

Graph 15b, showing the individual drug release for the 4 replicas shows that there was very low variability between replications. This indicates the robustness of the drug release.

Graph 16 shows that there was no relationship between in vivo and in vitro release of Kaolin formulation from the rumen delivery device.

As can be seen from Graph 16, in vitro release of Kaolin was quicker than that observed for in vivo.

### 2.6.2 In Vivo Drug Release Kaolin Formulation (200 Days)

Drug release of the individual 2 or 4 replicates, mean drug release and standard deviation is shown in Table 9.

TABLE 9
In vivo release of Kaolin (200 days)

| Time | Replicate 1 (% | Replicate 2 (% | Replicate 3 (% | Replicate 4 (% | Mean (% | |
|---|---|---|---|---|---|---|
| (weeks) | drug release) | drug release) | drug release) | drug release) | drug release) | SD |
| 0 | 0.0 | 0.0 | 0.0 | 0.0 | 0.0 | 0.0 |
| 4 | 12.1 | 15.1 | — | — | 13.6 | 2.1 |
| 8 | 27.7 | 31.8 | 27.8 | 28.5 | 29.0 | 2.0 |

Graphs 17 shows diagrammatically the in vitro release of Kaolin.

Graph 17a and b indicate that a linear release rate was observed for the first eight weeks of the trial. The drug release also appears to be fairly robust.

Graph 18 shows that there was no relationship between in vivo and in vitro release of Kaolin formulation from the rumen delivery device.

### 2.6.3 Kaolin Formulation (100 Day)—Observations:

It was observed that there was the formation of hollow spaces within the hydrogel/active component tablet.
This indicates that the rate of erosion was greater than the rate of swelling, with erosion taking place inside the rigid housing, instead of only outside as the hydrogel and active components are pushed out through the apertures. Therefore, the drug release was no longer controlled by the constant surface of the apertures. Observations are shown in Graph 19. A, B and C of Graph 19 show the observations for 2, 4 and 8 weeks respectively.
This behavior, along with the non-linear release rate at 100 days was not anticipated, the reasons, and ways to overcome this problem are discussed below.

### 2.6.4 Kaolin Formulation (200 Day)—Observations:

It was observed that there was the formation of hollow spaces within the hydrogel/active component tablet.
This indicates that the rate of erosion was greater than the rate of swelling, with erosion taking place inside the rigid housing, instead of only outside as the hydrogel and active components are pushed out through the apertures. Therefore, the drug release was no longer controlled by the constant surface of the apertures. Observations are shown in Graph 20. A and B of Graph 20 show the observations for 4 and 8 weeks respectively.
This behavior, along with the non-linear release rate at 100 days was not anticipated, the reasons, and ways to overcome this problem are discussed below.

### 2.6.5 Comparison of In Vitro Performance of Kaolin and Na Salicylate:

The swelling performance of the same formulations (both 20% PEO, 30% lactose and 50% drug) was compared. It was observed that quite different swelling occurred, this is shown in Graph 21 and 22 which show the swelling behavior of Kaolin and Na salicylate respectively.

As can be seen from Graphs 21 and 22, Kaolin showed no swelling of the hydrogel/active component out of the rigid housing through the apertures. In comparison, as desired Na salicylate shows considerable swelling of the hydrogel/active component out the rigid housing through the apertures—as expected.

This shows that the drug choice significantly impacts on the swelling of the hydrogel or PEO matrix.

The applicants believe the above difference in swelling between formulations containing Kaolin and Na salicylate may be due to the gel formation of PEO, and the interaction between PEO and the drug used.

The following interactions are believed to be the reason for the unexpected results when Kaolin (at 50%) was used.
Na-salicylate forms H-bonds with the ether oxygen of the PEO macromolecular chains (cross-linking). These bonds are assumed to support gel formation, as shown in Graph 23.
Kaolin (H₂Al₂Si₂O₈.H₂O) and MgSO₄ are supposed to reduce the molecular interactions between the PEO chains

### 2.6.6 Solution to Problem of Non Swelling of Kaolin/PEO Formulation

The lack of swelling is believed to be due to a lack of cross-linking when the PEO gels, or a weak gelling of same. It was anticipated that increasing the concentration of PEO should therefore overcome this problem. To test this the in vitro swelling of Kaolin with 20% and 40% PEO respectively were compared.

Increasing the PEO concentration to 40% apparently results in a ‘stronger’ gelling, and swelling of the hydrogel/active component through the apertures in the rigid housing was observed when a 40% concentration of PEO was utilized, as shown in Graphs 24 and 25.

40% PEO is assumed to provide a rate of swelling which is greater than the rate of erosion, so that erosion inside the device is prevented.

As then the aperture size determines the erosion surface, a linear drug release is expected.

### 2.6.7 Conclusions: Kaolin Formulation—100 and 200 Days

Kaolin is assumed to reduce the molecular interactions between the PEO chains (no cross-linking), resulting in a ‘fragile’ gel with 20% PEO where the rate of erosion was greater than the rate of swelling.
A new variant with 40% PEO will be tested in the animal trial to determine whether (as predicted) the rate of swelling will be greater than the rate of erosion.
   2.7 MgSO₄ Formulation (without HPMC)—100 Day (Variant #1)
   2.7.1 In Vivo Drug Release of MgSO₄ Formulation (without HPMC) (100 Days)

Drug release of the individual 4 replicates, mean drug release and standard deviation is shown in Table 10.

TABLE 10
In vivo release of MgSO₄ formulation (without HPMC) (100 days)

| Time | Replicate 1 (% | Replicate 2 (% | Replicate 3 (% | Replicate 4 (% | Mean (% | |
|---|---|---|---|---|---|---|
| (weeks) | drug release) | drug release) | drug release) | drug release) | drug release) | SD |
| 0 | 0.0 | 0.0 | 0.0 | 0.0 | 0.0 | 0.0 |
| 2 | −1.3 | −0.9 | −0.6 | 0.1 | −0.6 | 0.6 |
| 4 | −2.0 | −2.3 | −2.0 | −1.6 | −2.0 | 0.3 |
| 8 | 1.0 | 0.6 | −2.3 | −1.9 | −0.6 | 1.7 |

Graph 26 show diagrammatically the in vitro release of MgSO₄ formulation (without HPMC).

Graph 26 shows no in vivo drug release at all. The in vitro data has not yet been analyzed but appears to indicate no or minor drug release only.

It is assumed that the 1 mm aperture is too small for penetration of rumen fluid.

2.7.2 MgSO₄ Formulation (without HPMC) (100 Day)—Observations:
The tablets appeared unchanged after 8 weeks in the rumen as can be seen in Graph 27. A, B and C of Graph 27 relate to observations at 2, 4 and 8 weeks respectively.
   2.7.3 Conclusions: MgSO₄ Formulation (without HPMC) (100 Days)
The 1 mm aperture was apparently too small for drug release to occur.
A variant will be tested with a 2 mm aperture and 40% PEO, as MgSO₄, like Kaolin is assumed to reduce the molecular interactions of the PEO chains resulting in a ‘fragile’ gel.
   2.8 MgSO₄ Formulation (with HPMC)—100 Day
   2.8.1 In Vivo Drug Release MgSO₄ Formulation (with HPMC) (100 Days)

Drug release of the individual 4 replicates, mean drug release and standard deviation is shown in Table 11.

TABLE 11
In vivo release of MgSO₄ formulation (with HPMC) (100 days) - variant # 2.

| Time | Replicate 1 (% | Replicate 2 (% | Replicate 3 (% | Replicate 4 (% | Mean (% | |
|---|---|---|---|---|---|---|
| (weeks) | drug release) | drug release) | drug release) | drug release) | drug release) | SD |
| 0 | 0.0 | 0.0 | 0.0 | 0.0 | 0.0 | 0.0 |
| 2 | 10.8 | 13.8 | 12.2 | 12.1 | 12.2 | 1.2 |
| 4 | 25.3 | 27.7 | 27.0 | 21.2 | 25.3 | 2.9 |
| 8 | 48.4 | 46.4 | 47.2 | 48.1 | 47.5 | 0.9 |

Graphs 28 and 29 show diagrammatically the in vivo release of MgSO₄ formulation (with HPMC).

Graph 28a shows that linear drug release was observed for the first eight weeks of the trial, as expected.

Graph 28b, showing the individual drug release for the 4 replicas shows that there was very low variability between replications. This indicates the robustness of the drug release.

From the extrapolation of data it appears that the goal will be achieved—being zero-order drug release over 16 weeks.

2.8.2 MgSO₄Formulation (with HPMC) (100 Days)—Observations:
No or only very slight hollow space formation within the rigid housing was observed. As can be seen in Graph 30. A, B and C of Graph 30 relate to observations at 2, 4 and 8 weeks respectively.
   2.8.3 Conclusions: MgSO₄ formulation (with HPMC) (100 Days)
So far, perfect zero order drug release was observed when HPMC was included in the formulation.
A 4 mm aperture instead of 3 mm will be tested as a new variant in vivo

### 2.9 Overall Conclusions:

The drug release looks very promising for both Na-salicylate variants and MgSO4 variant with HPMC
A 1 mm hole is assumed to be too small for rumen fluid to penetrate in the device.
The drug type at high drug load significantly affects the gel formation/swelling of the matrix:
Organic molecules with H-bond donator functional groups (amide, —OH, phenolic groups, amines, —COOH) are assumed to support the gel formation by cross-linking the PEO chains. Lower contents of PEO provide a sufficiently stable gel for a controlled drug release (rate of erosion controlled by the constant surface of the holes)
Molecules without H-bond donator functional groups (e.g. inorganic minerals) are assumed to reduce the interactions between the PEO macromolecules. Higher contents of PEO are needed to provide a sufficiently stable gel for a controlled drug release.
For low drug loads (<20%), no or minor impact of drug type on swelling behaviour of the matrix is anticipated

Aspects of the present invention have been described by way of example only and it should be appreciated that modifications and additions may be made thereto without departing from the scope thereof as defined in the appended claims.

1. A control release device for the delivery of active components, the device including;
a substantially impermeable rigid housing containing at least one discrete aperture therein,
a driving substance containing at least one active component placed within the housing,
characterised in that the driving substance swells in the presence of fluid, driving the substance and active component(s) out of the housing through the aperture(s).

2. A control release device as claimed in claim 1 wherein the device is used for the delivery of at least one active component to an environment which contains a fluid capable of activating the driving substance.

3. A control release device as claimed in claim 1 wherein the device is for administration to the digestive system, or rumen of an animal.

4. A control release device as claimed in claim 1 wherein the housing is impermeable to fluid, except through the aperture(s).

5. A control release device as claimed in claim 1, wherein the housing is configured to break space down internally after substantially all the active component(s) have been released.

6. A control release device as claimed in claim 1 wherein the housing is configured to be excreted by the animal after substantially all the active component(s) have been released.

7. A control release device as claimed in claim 1, wherein the housing is substantially cylindrical in shape.

8. A control release device as claimed in claim 1, wherein the housing also includes at least one wing.

9. A control release device as claimed in claim 1, wherein the housing contains at least one aperture along the longitudinal sides of the housing.

10. A control release device as claimed in claim 1, wherein the housing is configured with at least one row of apertures along the longitudinal sides of same.

11. A control release device as claimed in claim 1, wherein the housing is configured with at least one aperture on at least one end of the housing.

12. A control release device as claimed in claim 1, wherein the active component is a component which has a beneficial action in the environment of use.

13. A control release device as claimed in claim 1, wherein the driving substance is a material which swells on contact with a fluid.

14. A control release device as claimed in claim 1, wherein the driving substance is at least one hydrogel.

15. A control release device as claimed in claim 14, wherein the driving substance is polyethylene oxide (PEO).

16. A control release device as claimed in claim 13, wherein the swollen driving substance undergoes dissolution in the presence of fluid.

17. A control release device as claimed in claim 1 wherein the driving substance is a compound which generates a gas when it comes into contact with a fluid.

18. A control release device as claimed in claim 1, wherein the driving substance containing at least one active component is in the form of a tablet.

19. A method of treating an animal with at least one active component via a control release device as claimed in claim 1, the method characterised by the step of
a) administering of the control release device to an animal, or other environment for use,
wherein the control release device includes
a substantially impermeable rigid housing containing at least one discrete aperture therein,
a driving substance containing at least one active component placed within the housing which swells in the presence of fluid, driving the substance and active compounds out of the housing through the aperture(s).

20. A method of manufacturing a control release device, characterized by the step of
a) placing a driving substance containing at least one active component into a substantially impermeable rigid housing, wherein the housing includes at least one discrete aperture therein,
wherein the driving substance swells in the presence of fluid, driving the substance and active compound(s) out of the housing through the aperture(s).

21. (canceled)

22. (canceled)

23. (canceled)
